(19) United States
(12) Patent Application Publication
(10) Pub. No.: US 2025/0001032 A1
(43) Pub. Date: Jan. 2, 2025

(54) SCENTED WAX WARMER

(71) Applicant: B&B Acquisition, Inc. DBA Candle Warmers, Etc., Draper, UT (US)

(72) Inventors: Brian K. BEESLEY, Draper, UT (US); Meghan SAUNDERS, Draper, UT (US)

(21) Appl. No.: 18/343,666

(22) Filed: Jun. 28, 2023

(51) Int. Cl. A61L 9/03

(52) U.S. Cl. CPC A61L 9/03; A61L 2209/12

(57) ABSTRACT

A wax warmer has a shroud removably carried by a base with a bowl for scented wax. A heat source is carried by the shroud. A proximal end of an electrical wire is coupled to the heat source. A wire box is carried by the shroud and positioned between the shroud and the heat source. The wire box contains an electrical connection between the proximal end of the electrical wire and the heat source. A clamp opening carried by the wire box receives the proximal end of the wire and presses against a sheath of the electrical wire to retain the proximal end of the electrical wire with the wire box.

### BACKGROUND

Wickless, scented candles utilize heat to melt a wax in order to release a fragrance. The fragrance can be aesthetically pleasing and may also have therapeutic value. The traditional wick can be replaced by an electric heat source. Such devices are often referred to as wax warmers. The heat source can be an incandescent or halogen light bulb. The light bulb can be placed underneath a vessel containing the scented wax. The light bulb may also be positioned above a vessel of scented wax. Light from the light bulb can also be utilized for aesthetics and/or utilitarian purposes, such as providing a lamp. The scented wax can be provided in a jar that is placed on or below the heat source. The scented wax can also be provided loose and can be added to an existing vessel. Improvement to wax warmers and wickless, scented candles is an ongoing endeavor.

### BRIEF DESCRIPTION OF THE DRAWINGS

Features and advantages of the invention will be apparent from the detailed description which follows, taken in conjunction with the accompanying drawings, which together illustrate, by way of example, features of the invention;

and, wherein:

FIG. 1 is front side view of a wax warmer in accordance with an embodiment of the invention.

FIG. 2 is a cross-sectional side view of the wax warmer of FIG. 1.

FIG. 3 is an exploded side view of the wax warmer of FIG. 1.

FIG. 4 is a cross-sectional side view of the wax warmer of FIG. 1.

FIG. 5 is a partial, detailed cross-sectional side view of the wax warmer of FIG. 1.

FIG. 6a is a partially exploded top perspective view of a wire box of the wax warmer of FIG. 1.

FIG. 6b is a partially exploded bottom perspective view of the wire box of the wax warmer of FIG. 1.

FIG. 7 is a cross-sectional side view of the wire box of the wax warmer of FIG. 1.

FIG. 8 is a perspective view of the wire box of the wax warmer of FIG. 1.

FIG. 9 is a top view of the wire box of FIG. 8.

FIG. 10 is a bottom view of the wire box of FIG. 8.

FIG. 11 is a side view of the wire box of FIG. 8.

FIG. 12 is a front view of the wire box of FIG. 8.

FIG. 13 is a cross-sectional side view of the wire box of FIG. 8.

FIG. 14 is a perspective view of a cover of the wire box of the wax warmer of FIG. 1.

FIG. 15 is a top view of the cover of FIG. 14.

FIG. 16 is a bottom view of the cover of FIG. 14.

FIG. 17 is a side view of the cover of FIG. 14.

FIG. 18 is a front view of the cover of FIG. 14.

FIG. 19 is a cross-sectional side view of the cover of FIG. 14.

FIG. 20 is a perspective view of a cap of the wax warmer of FIG. 1.

FIG. 21 is a top view of the cap of FIG. 20.

FIG. 22 is a bottom view of the cap of FIG. 20.

FIG. 23 is a side view of the cap of FIG. 20.

FIG. 24 is a front view of the cap of FIG. 20.

FIG. 25 is a cross-sectional side view of the cap of FIG. 20.

Reference will now be made to the exemplary embodiments illustrated, and specific language will be used herein to describe the same. It will nevertheless be understood that no limitation of the scope of the invention is thereby intended.

### DETAILED DESCRIPTION

Before invention embodiments are disclosed and described, it is to be understood that no limitation to the particular structures, process steps, or materials disclosed herein is intended, but also includes equivalents thereof as would be recognized by those ordinarily skilled in the relevant arts. It should also be understood that terminology employed herein is used for the purpose of describing particular examples only and is not intended to be limiting. The same reference numerals in different drawings represent the same element. Numbers provided in flow charts and processes are provided for clarity in illustrating steps and operations and do not necessarily indicate a particular order or sequence. Unless defined otherwise, all technical and scientific terms used herein have the same meaning as commonly understood by one of ordinary skill in the art to which this disclosure belongs.

An initial overview of the inventive concepts are provided below and then specific examples are described in further detail later. This initial summary is intended to aid readers in understanding the examples more quickly, but is not intended to identify key features or essential features of the examples, nor is it intended to limit the scope of the claimed subject matter.

A wax warmer can be provided for warming a scented wax in order to release a fragrance and light for aesthetic and/or therapeutic purposes. The scented was may also include an oil, such as an essential oil. The wax warmer can be a top-down warmer in which heat is applied above the wax and directed downward towards the wax. The wax warmer can have a shroud that at least partially covers a base containing the scented wax. The shroud can carry and position a heat source above the base and the wax. The shroud can have at least one aperture in one aspect, and a plurality or array of apertures in another aspect, to release scent and/or light from the shroud and the base. The wax warmer and the base can be positioned on a support surface, such as a counter, desk or shelf. The scented wax can be added to the base by lifting the shroud off of the base to expose the base. The heat source can be a light bulb powered by electricity through an electrical cord extending from the wax warmer.

The shroud can comprise and can be formed of a ceramic material for structural and aesthetic purposes. The material of the shroud can be brittle and may be prone to breaking if dropped. The material of the shroud can present challenges for electrical components carried by and/or contained in the shroud. The aesthetic and therapeutic nature of the fragrance/aroma and the light can provide structural challenges. The present wax warmer can be configured to protect the shroud from breakage, and/or to contain electrical component should such breakage occur.

In one aspect, a proximal end of the electrical cord can be contained in a wire box so that electrical connections between the cord and a socket of the heat source are contained within the box without being exposed. The wire box can be formed of plastic that can be less brittle than the shroud. The wire box can also have a clamp opening to clamp the proximal end of the cord to prevent or resist separation of the proximal end of the cord from the socket and the wire box; thus resisting exposure.

In another aspect, the electrical cord can be positioned to enter the shroud near a bottom, and extend to the top of the shroud. Thus, the cord can be positioned closer to the support surface during use to resist interference and inadvertent engagement. In addition, the cord can extend through multiple bores in the shroud in a serpentine path to further resist separation of the cord from the shroud, the wire box and the socket.

In another aspect, the heat source and the wire box can be pendant from a cap on top of the shroud. The wire box can be coupled to the cap through a hole in the top of the shroud. The cap can distribute force across a broader area of the shroud and reduce stress concentration at the hole to resist breakage.

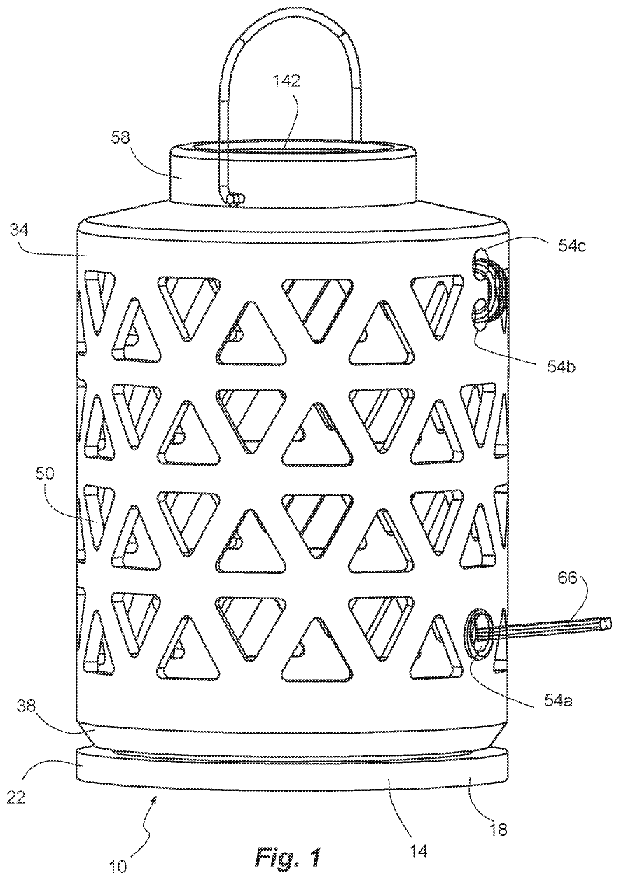

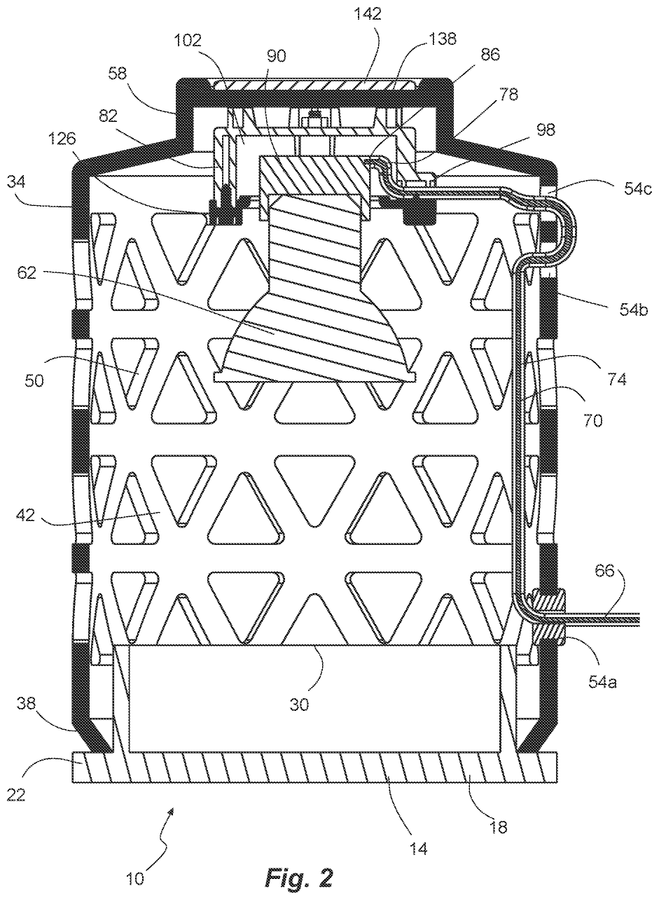

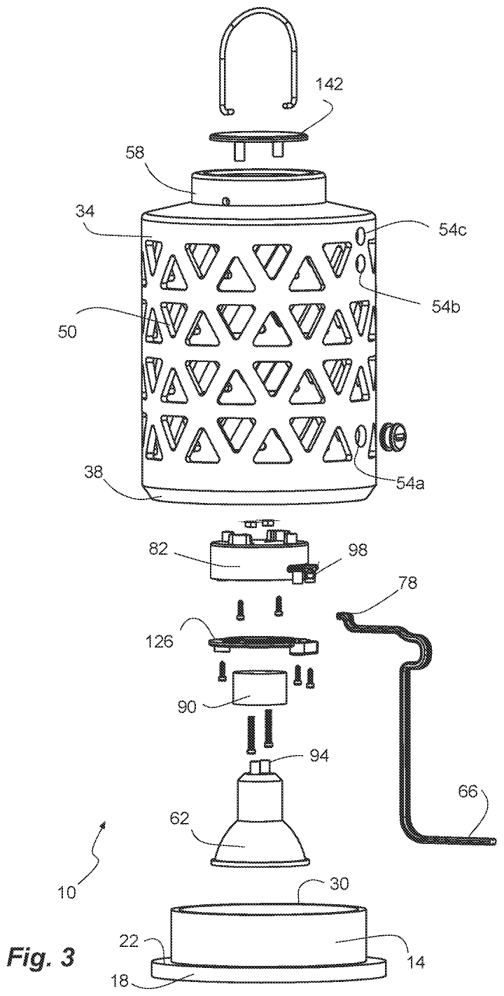

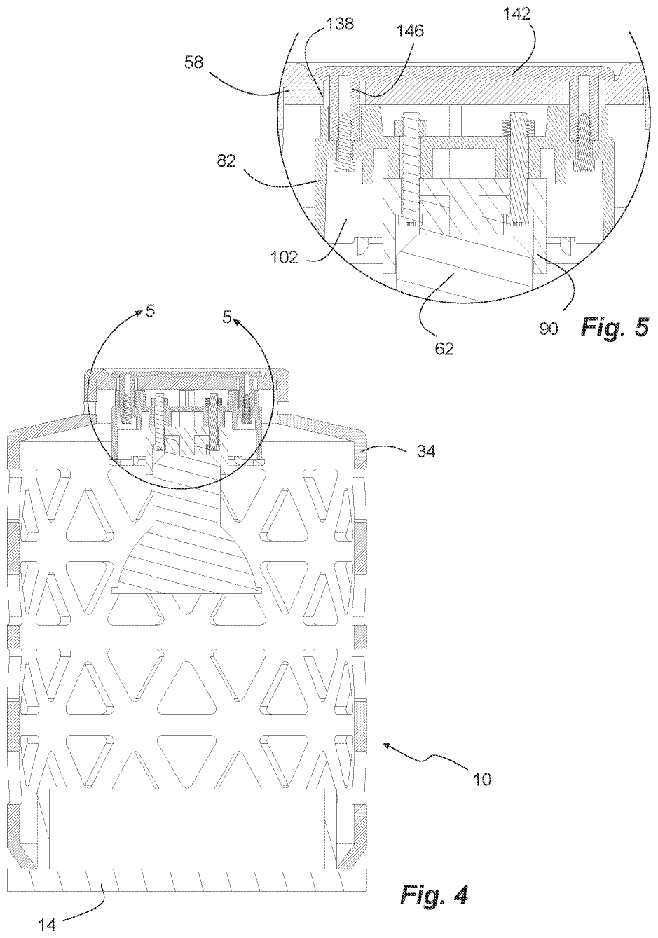

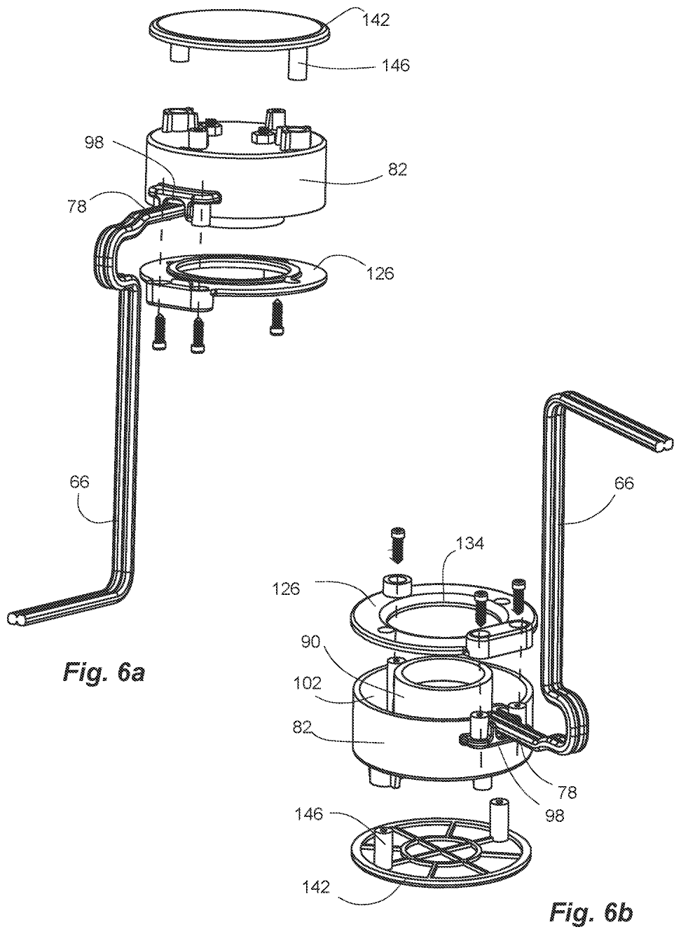

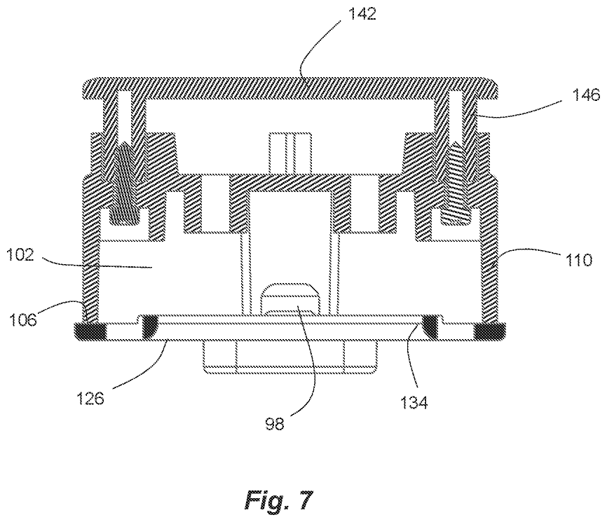

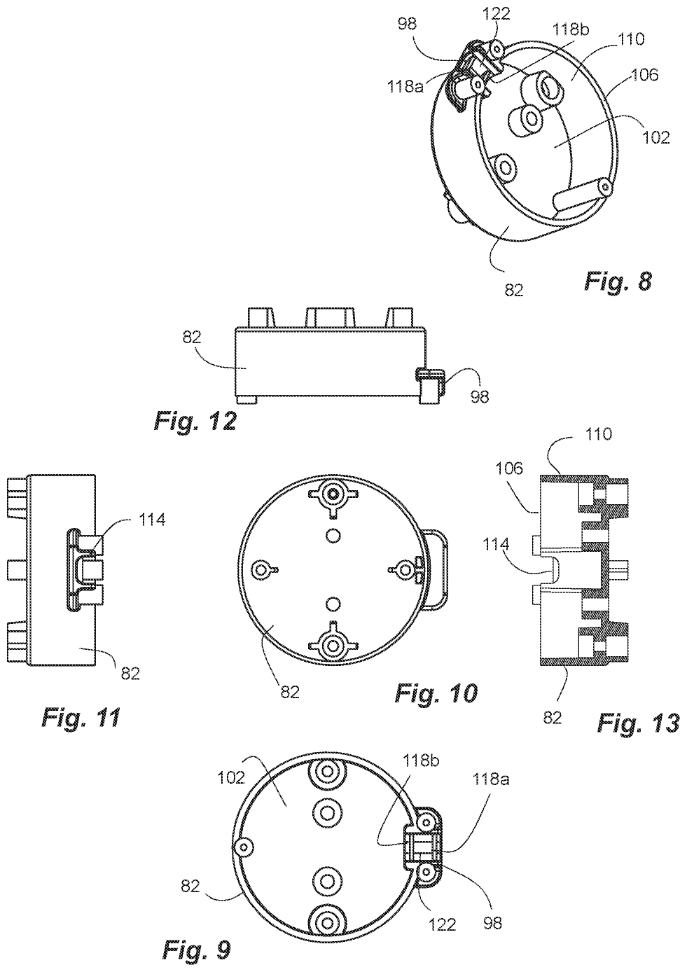

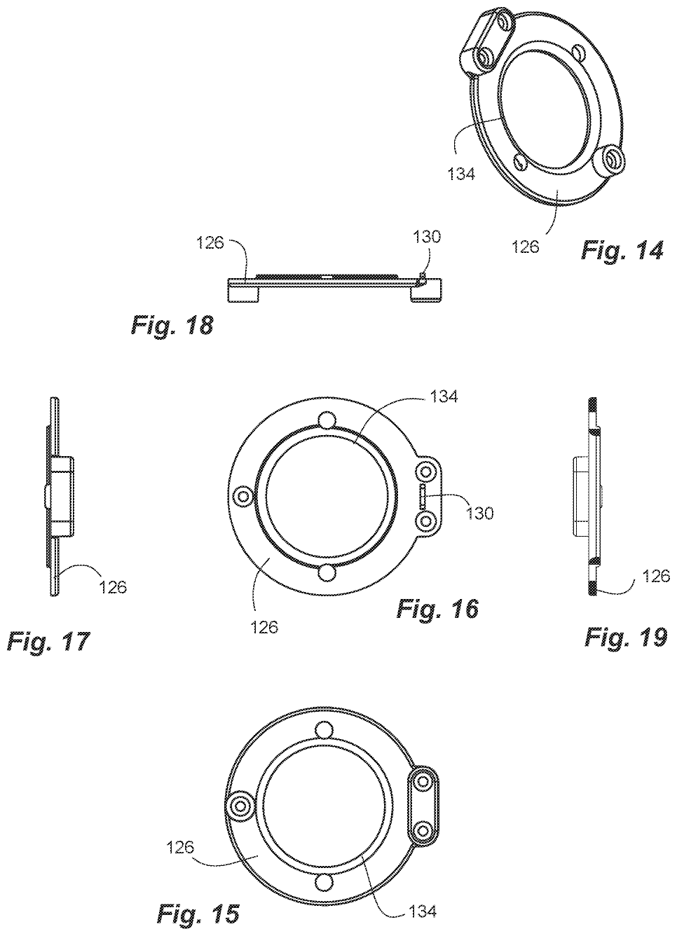

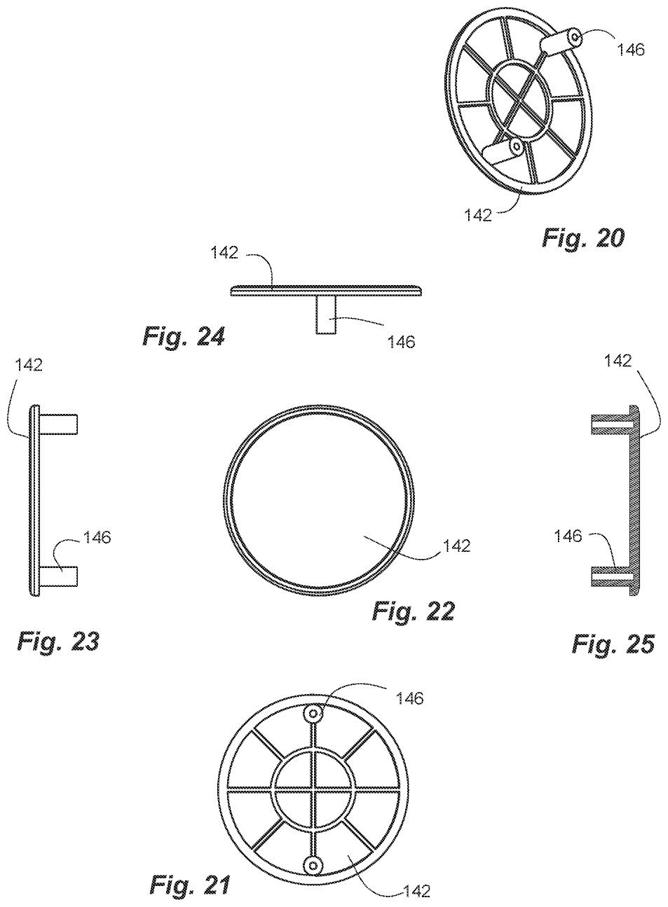

Referring to FIGS. 1-25, a wax warmer 10 or candle warmer lamp in an example of the invention is shown. The wax warmer 10 can warm a scented wax to release fragrance from the wax into the air. The fragrance can be aesthetically pleasing and/or therapeutic. In addition, the wax warmer 10 can provide ambiance with its physical presentation and/or by emitting light. The wax warmer can be wickless and can provide electric heating.

The wax warmer 10 can have a base 14 that can be placed on a support surface, such as a counter, shelf or desk. The base 14 can have a plate 18 with a flat bottom and an annular lip 22 circumscribing the plate 18. A bowl 26 can extend from the plate 18 and the annular lip 22. Thus, the annular lip 22 can also circumscribe the bowl 26 and extend beyond a perimeter of the bowl 26. The bowl 26 can have an open top 30 and can receive and contain the scented wax. The base 14 can comprise or be formed of ceramic.

A shroud 34 can be removably carried by the base 14. The shroud 34 can have an open bottom 38 that can be received on the annular lip 22 of the base 14. The shroud 34 can also have an interior 42 with the bowl 26 received through the open bottom 38 and into the interior 42. The shroud 34 can be placed on the base 14 during operation and use, and removed from the base 14 to add the scented wax to the bowl 26.

The shroud 34 can have a perimeter wall 46 with at least one aperture 50. In another aspect, the perimeter wall 46 can have a plurality or an array of apertures 50. Thus, the shroud 34 can at least partially obscure the bowl 26 and the wax while allowing fragrance to exit the interior 42 of the shroud 34.

The shroud 34 can also have at least one bore 54a therethrough for an electrical cord, as described herein. The shroud 34 and the perimeter wall 46 can also have a top 58 opposite the bottom opening 38. The shroud 34 can also comprise or be formed of ceramic. The ceramic material can be more brittle than plastic.

A heat source 62 can be carried by the shroud 34 and positioned in the interior 42 of the shroud 34. In one aspect, the heat source 62 can be a 25 watt, 110-130 volt, halogen or incandescent light bulb. The heat source 62 can be pendant from the top 58 of the shroud 34 and positioned above the bowl 26. The head source 62 and the light bulb can be directional, such as with a conical or parabolic reflector. Heat from the heat source 62 and the light can be directed towards the bowl 26 and thus towards the scented wax in order to melt the wax and release the fragrance.

An electrical wire or cord 66 can extend through the bore 54a in the shroud 34 and can be electrically coupled to the heat source 62. The electrical wire 66 can have a conductive core 70 in an exterior insulative sheath 74. The electrical wire 66 can have a proximal end 78 with exposed conductive cores.

A wire box 82 can be carried by the top 58 of the shroud 34. The wire box 82 can be positioned between the shroud 34 and the heat source 62. Thus, the wire box 82 can be positioned nearer the top 58 than the open bottom 38 of the shroud 34. The wire box 82 can enclose an electrical connection 86 between the proximal end 78 of the electrical wire 66 and the heat source 62.

A socket 90 can be carried by the wire box 82 and pendent from the top 58 of the shroud 34. An end of the light bulb 62 can be removably received in the socket 90. The light bulb 62 and the socket 90 can have mating terminals 94. In one aspect, the exposed conductive cores of the wire 66 can be coupled to terminals of the socket 90. The wire box 82 can surround the exposed conductive cores of the proximal end 78 of the wire 66 and the electrical connection 86.

A clamp opening 98 can be associated with and carried by the wire box 82. The clamp opening 98 can receive the proximal end 78 of the wire 66. The clamp opening 98 can press against the sheath 74 of the electrical wire 66 and retain the proximal end 78 of the electrical wire 66 with the wire box 82. The proximal end 78 of the electrical wire 66 can be positively retained and contained within the wire box 82. The wire box 82 can comprise or can be formed of plastic.

In one aspect, the wire box 82 can have a cavity 102 with an opening 106 surrounded by a wall 110. A notch 114 can be formed in the wall 110 of the wire box 82 proximate the opening 106. A pair of spaced-apart tabs 118a-b can be formed in the notch 114 and arranged in series along an axis of the notch 114. A gap 122 can be formed between the pair of spaced-apart tabs 118a-b.

The wire box 82 can also have a cover 126 coupled to the opening 106 of the wire box 82. An opposing tab 130 can extend from the cover 126 and can be aligned with the gap 122 between the pair of spaced-apart tabs 118a-b in the notch 114. The tabs 118a-b and 130 of the wire box 82 and the cover 126 can form the clamp opening 98 of the wire box 82 with the tabs 118a-b and 130 bearing against the sheath 74 of the electrical wire 66.

The wire box 82 and the cover 126 can have an opening 134 corresponding to the socket 90. Thus, the end of the light bulb 62 can extend through the opening 134 in the cover 126 and into the socket 90. The wire box 82 can completely surround the socket 90 except for the opening 134 corresponding to the socket 90.

As described herein, the shroud 34 can be formed of a ceramic material while the wire box 82 can be formed of a plastic material. Thus, the shroud 34 can be more brittle than the wire box 82. If the shroud 34 is broken, only the wire box 82 inside the interior of the shroud 34 is exposed, while the proximal end 78 of the wire 66, the exposed conductive cores and the electrical connection 86 are maintained inside the wire box 82 to avoid contact and electric shock. In addition, the clamp opening 98 and the tabs 118a-b and 130 thereof can resist separation of the wire 66 from the wire box 82 to further avoid electric shock.

As described herein, the shroud 34 can have at least one bore 54a. In another aspect, the shroud 34 can have at least three bores 54a-c, including: a bottom bore 54a closer to the open bottom 38 of the shroud 34, and first and second top bores 54b-c closer to the top 58 of the shroud 34. The electrical wire 66 can extend in a serpentine path into and out of the shroud 34. The electrical wire 66 can extend into the shroud 34 through the bottom bore 54a, extend up the interior 42 of the shroud 34 proximate the perimeter wall 46 of the shroud 34, extend out of the shroud 34 through the first top bore 54b, and extend back into the shroud 34 through the second top bore 54c. Threading the wire 66 through the shroud 34 multiple times can further resist separation of the proximal end 78 of the wire 66 from the wire box 82 by providing additional resistance between the wire 66 and the shroud 34. In addition, the wire 66 is provided closed to a bottom of the shroud 34, and thus the support surface, to avoid unintentional interference.

As described herein, the shroud 34 can have at least one hole 138 through the top 58 of the shroud 34. In another aspect, the shroud 34 can have a pair of holes 138 through the top 58 of the shroud 34. A cap 142 can be carried by the top 58 of the shroud 34 and positioned outside the shroud 34 above hole(s) 138. The cap 142 can abut to an exterior of the shroud 34 beyond the at least one hole(s) 138. The wire box 82, the socket 90 and the light bulb 62 can be pendant from the cap 142 through the hole(s) 138 in the shroud 34. The wire box 82 can be coupled to the cap 142 through the hole(s) 138 and pendent from the cap 142. In one aspect, at least one ferrule 146, or a pair of ferrules, can extend from the cap 142 and through the hole(s) 138. The wire box 82 can be coupled to the cap 142 by the ferrule(s) 146, such as with threaded fasteners. Thus, a portion of the shroud 34 can be held between the cap 142 and the wire box 82. Suspending the wire box 82, the socket 90 and the light bulb 62 from the cap 142 can reduce stress concentration on the shroud 34 and at the hole(s) 138 to avoid breakage of the shroud 34.

As used in this specification and the appended claims, the singular forms “a,” “an” and “the” include plural referents unless the context clearly dictates otherwise. Thus, for example, reference to “a layer” includes a plurality of such layers.

In this disclosure, “comprises,” “comprising.” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term in the specification, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top.” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility to a numerical range endpoint by providing that a given value may be “a little above” or “a little below” the endpoint. It is understood that express support is intended for exact numerical values in this specification, even when the term “about” is used in connection therewith.

It is to be understood that the examples set forth herein are not limited to the particular structures, process steps, or materials disclosed, but are extended to equivalents thereof as would be recognized by those ordinarily skilled in the relevant arts. It should also be understood that terminology employed herein is used for the purpose of describing particular examples only and is not intended to be limiting.

Furthermore, the described features, structures, or characteristics may be combined in any suitable manner in one or more examples. In the description, numerous specific details are provided, such as examples of lengths, widths, shapes, etc., to provide a thorough understanding of the technology being described. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the invention in one or more particular applications, it will be apparent to those of ordinary skill in the art that numerous modifications in form, usage and details of implementation can be made without the exercise of inventive faculty, and without departing from the principles and concepts described herein. Accordingly, it is not intended that the invention be limited, except as by the claims set forth below.

What is claimed is:

1. A wax warmer device configured to warm a scented wax, the device comprising:
a base having a plate with an annular lip circumscribing the plate and a bowl extending from the plate and the annular lip, the bowl having an open top and being configured to receive and contain the scented wax;
a shroud removably carried by the base and having an open bottom receivable on the annular lip of the base and an interior with the bowl receivable through the open bottom and into the interior, the shroud having a perimeter wall with at least one aperture and at least partially obscuring the bowl, the shroud having a bore therethrough;
a heat source carried by the shroud and positioned in the interior of the shroud, the heat source being pendant from the shroud and positionable above the bowl;
an electrical wire extending through the bore in the shroud and electrically coupled to the heat source, the electrical wire having a conductive core in an exterior insulative sheath, the electrical wire having a proximal end;
a wire box carried by the shroud and positioned between the shroud and the heat source, and containing an electrical connection between the proximal end of the electrical wire and the heat source; and
a clamp opening carried by the wire box and receiving the proximal end of the wire, the clamp opening pressing against the sheath of the electrical wire and retaining the proximal end of the electrical wire with the wire box, and wherein the proximal end of the electrical wire is positively retained and contained within the wire box.

2. The wax warmer device in accordance with claim 1, further comprising:
the wire box having a cavity with an opening surrounded by a wall;
a notch in the wall of the wire box;
a pair of spaced-apart tabs in the notch and arranged in series along an axis of the notch;
a gap between the pair of spaced-apart tabs;
a cover coupled to the opening of the wire box;
an opposing tab extending from the cover and aligned with the gap between the pair of spaced-apart tabs in the notch;
the tabs of the wire box and the cover forming the clamp opening of the wire box with the tabs bearing against the sheath of the electrical wire.

3. The wax warmer device in accordance with claim 1, further comprising:
the shroud formed of a ceramic material;
the wire box formed of a plastic material; and
the shroud being more brittle than the wire box.

4. The wax warmer device in accordance with claim 1, further comprising:
a socket carried by the wire box;
the heat source comprising a light bulb with an end removably received in the socket;
the wire box having an opening corresponding to the socket; and
the wire box completely surrounding the socket except for the opening corresponding to the socket.

5. The wax warmer device in accordance with claim 1, further comprising:
at least one hole through the shroud;
a cap carried by the shroud and positioned outside the shroud at the at least one hole; and
the wire box coupled to the cap through the at least one hole and pendent from the cap.

6. The wax warmer device in accordance with claim 5, further comprising:
the cap abutting to an exterior of the shroud beyond the at least one hole; and
a portion of the shroud held between the cap and the wire box.

7. The wax warmer device in accordance with claim 5, further comprising:
at least one ferrule extending from the cap and through the at least one hole; and
the wire box coupled to the cap by the at least one ferrule.

8. The wax warmer device in accordance with claim 5, further comprising:
the shroud having a top opposite the open bottom;
the at least one hole extending through the top of the shroud; and
the cap positioned at the top of the shroud.

9. The wax warmer device in accordance with claim 1, further comprising:
the shroud having a top opposite the open bottom;
the wire box positioned nearer the top than the open bottom of the shroud;
the bore comprising at least three bores including a bottom bore closer to the open bottom of the shroud and first and second top bores closer to the top of the shroud; and
the electrical wire extending in a serpentine path into the shroud through the bottom bore, extending up the interior of the shroud proximate the perimeter wall of the shroud, extending out of the shroud through the first top bore, and extending back into the shroud through the second top bore.

10. A wax warmer device configured to warm a scented wax, the device comprising:
a base having a plate with an annular lip circumscribing the plate and a bowl extending from the plate and the annular lip, the bowl having an open top and being configured to receive and contain the scented wax;
a shroud removably carried by the base and having an open bottom receivable on the annular lip of the base and an interior with the bowl receivable through the open bottom and into the interior, the shroud having a perimeter wall with at least one aperture and at least partially obscuring the bowl, the shroud having a top;
a heat source carried by the shroud and positioned in the interior of the shroud closer to the top than the open bottom, the heat source being pendant from the shroud and positionable above the bowl;
an electrical wire electrically coupled to the heat source;
at least three bores extending through the shroud including a bottom bore closer to the open bottom of the shroud and first and second top bores closer to the top of the shroud; and
the electrical wire extending in a serpentine path into the shroud through the bottom bore, extending up the interior of the shroud proximate the perimeter wall of the shroud, extending out of the shroud through the first top bore, and extending back into the shroud through the second top bore.

11. The wax warmer device in accordance with claim 10, further comprising:
the electrical wire having a conductive core in an exterior insulative sheath, the electrical wire having a proximal end;
a wire box carried by the shroud and positioned between the shroud and the heat source, and containing an electrical connection between the proximal end of the electrical wire and the heat source; and
a clamp opening carried by the wire box and receiving the proximal end of the wire, the clamp opening pressing against the sheath of the electrical wire and retaining the proximal end of the electrical wire with the wire box, and wherein the proximal end of the electrical wire is positively retained and contained within the wire box.

12. The wax warmer device in accordance with claim 11, further comprising:
the wire box having a cavity with an opening surrounded by a wall;
a notch in the wall of the wire box;
a pair of spaced-apart tabs in the notch and arranged in series along an axis of the notch;
a gap between the pair of spaced-apart tabs;
a cover coupled to the opening of the wire box;
an opposing tab extending from the cover and aligned with the gap between the pair of spaced-apart tabs in the notch;
the tabs of the wire box and the cover forming the clamp opening of the wire box with the tabs bearing against the sheath of the electrical wire.

13. The wax warmer device in accordance with claim 11, further comprising:
the shroud formed of a ceramic material;
the wire box formed of a plastic material; and
the shroud being more brittle than the wire box.

14. The wax warmer device in accordance with claim 11, further comprising:
a socket carried by the wire box;
the heat source comprising a light bulb with an end removably received in the socket;
the wire box having an opening corresponding to the socket; and
the wire box completely surrounding the socket except for the opening corresponding to the socket.

15. The wax warmer device in accordance with claim 11, further comprising:
at least one hole through the shroud;
a cap carried by the shroud and positioned outside the shroud at the at least one hole; and
the wire box coupled to the cap through the at least one hole and pendent from the cap.

16. The wax warmer device in accordance with claim 15, further comprising:
the cap abutting to an exterior of the shroud beyond the at least one hole; and
a portion of the shroud held between the cap and the wire box.

17. The wax warmer device in accordance with claim 15, further comprising:
at least one ferrule extending from the cap and through the at least one hole; and
the wire box coupled to the cap by the at least one ferrule.

18. The wax warmer device in accordance with claim 15, further comprising:
the at least one hole extending through the top of the shroud; and
the cap positioned at the top of the shroud.

19. A wax warmer device configured to warm a scented wax, the device comprising:
a base having a plate with an annular lip circumscribing the plate and a bowl extending from the plate and the annular lip, the bowl having an open top and being configured to receive and contain the scented wax;
a shroud removably carried by the base and having an open bottom receivable on the annular lip of the base and an interior with the bowl receivable through the open bottom and into the interior, the shroud having a perimeter wall with at least one aperture and at least partially obscuring the bowl, the shroud having a top opposite the open bottom;
a heat source carried by the shroud and positioned in the interior of the shroud, the heat source being pendant from the shroud and positionable above the bowl;
an electrical wire electrically coupled to the heat source, the electrical wire having a conductive core in an exterior insulative sheath, the electrical wire having a proximal end;
a wire box carried by the shroud and positioned between the shroud and the heat source, and containing an electrical connection between the proximal end of the electrical wire and the heat source;
a clamp opening carried by the wire box and receiving the proximal end of the wire, the clamp opening pressing against the sheath of the electrical wire and retaining the proximal end of the electrical wire with the wire box, and wherein the proximal end of the electrical wire is positively retained and contained within the wire box;
at least three bores extending through the shroud including a bottom bore closer to the open bottom of the shroud and first and second top bores closer to the top of the shroud; and
the electrical wire extending in a serpentine path into the shroud through the bottom bore, extending up the interior of the shroud proximate the perimeter wall of the shroud, extending out of the shroud through the first top bore, and extending back into the shroud through the second top bore.

20. The wax warmer device in accordance with claim 19, further comprising:
at least one hole through the top of the shroud;
a cap carried by the shroud and positioned at the top of the shroud at the at least one hole;
the cap abutting to an exterior of the shroud beyond the at least one hole;
the wire box coupled to the cap through the at least one hole and pendent from the cap; and
a portion of the shroud held between the cap and the wire box.
